(19) United States
(12) Patent Application Publication
(10) Pub. No.: US 2011/0015613 A1
(43) Pub. Date: Jan. 20, 2011

(54) Medical Blocking Tool

(75) Inventor: Takao Anzai, Kanagawa (JP)

Correspondence Address: BUCHANAN, INGERSOLL & ROONEY PC, POST OFFICE BOX 1404, ALEXANDRIA, VA, 22313-1404, US

(73) Assignee: TERUMO KABUSHIKI KAISHA, Tokyo (JP)

(21) Appl. No.: 12/893,098

(22) Filed: Sep. 29, 2010

Related U.S. Application Data
(63) Continuation of application No. PCT/JP2009/055857, filed on Mar. 24, 2009.

(30) Foreign Application Priority Data
Mar. 31, 2008 (JP) 2008-093548

(51) Int. Cl. A61M 31/00

(52) U.S. Cl. 604/514

(57) ABSTRACT

A medical blocking tool includes a core section and a swelling section provided around the core section. The core section reinforces the swelling section. The swelling section is formed of a swelling material which swells (expands) by absorbing a liquid so that the volume of the swelling section increases. The swelling section is adapted to swell until a bronchial tube is blocked while coming into close contact with the inner wall of the bronchial tube at a portion to be blocked (a blocking target portion) in the bronchial tube.

This application is a continuation of International Application No. PCT/JP2009/055857 filed on Mar. 24, 2009, and claims priority to Japanese Application No. 2008-093548 filed on Mar. 31, 2008, the entire content of both of which is incorporated herein by reference

### TECHNICAL FIELD

The present invention generally pertains to a medical blocking tool. More specifically, the invention relates to a medical blocking tool configured to be placed inside a lumen of a body to block the lumen.

### BACKGROUND DISCUSSION

Emphysema is a lesion which is oftentimes formed by the inhalation of harmful substances during smoking or the like, and causes a wide range of damage to the alveoli and distal airways of the lungs. The formation of the lesion is chronically progressive, and the respiratory function of a patient having such a chronic progressive lesion is remarkably inhibited.

The main therapies for a patient having such emphysema are largely classified into medical therapy and surgical therapy. Medical therapies tend to relieve the patient's symptomatic state, but do not do much to stop the progression of the emphysema.

Examples of surgical therapies include lung volume reduction surgery and lung transplant. All of the therapies require patients to undergo a major procedure, and this places an extraordinarily large burden on the patient and also requires expenditure of a huge amount of money. For these reasons, surgical therapies cannot be easily carried out.

A medical blocking tool which is not so burdensome to the patient and not so expensive and can be used in therapy for emphysema is described in Japanese Application publication No. 2004-24864. This application publication describes a resinous bronchial tube blocking material (blocking material) which is formed in a conical trapezoid shape. The blocking material is placed in a predetermined portion of a bronchial tube by the use of a bronchoscope (an endoscope for a bronchial tube) or the like to block the predetermined portion of the bronchial tube, where the outer diameter of the blocking material is set to be larger than the inner diameter of the portion (blocking target portion) to be blocked in the bronchial tube.

Because the outer diameter of the blocking material is larger than the inner diameter of the lumen of the bronchoscope, the blocking material cannot be inserted into the lumen of the bronchoscope. For this reason, the blocking material is gripped at the front end side of the bronchoscope by a clamp or the like inserted through the lumen of the bronchoscope and protruding outward from the front end of the bronchoscope, and is transported and placed at the blocking target portion of the bronchial tube together with the bronchoscope.

However, because the outer diameter of the blocking material of the related art is larger than the inner diameter of the blocking target portion of the bronchial tube, and the blocking material is gripped at the front end side of the bronchoscope by the use of the clamp to be transported and placed at the blocking target portion, many years of experience and a relatively high skill level are required to perform the treatment.

In addition, the blocking material is generally placed at each of a plurality of positions, but whenever the placement for each blocking material is completed, it is necessary to perform a series of operations whereby the bronchoscope is extracted from inside the body, the subsequent blocking material to be placed is gripped by the clamp protruding outward from the front end of the bronchoscope, and then the bronchoscope is re-inserted into the body. For this reason, the treatment time is lengthy, and the burden on the patient is exceedingly large.

### SUMMARY

The medical blocking tool disclosed here can be relatively quickly, easily, and reliably placed inside the lumen of a body.

The medical blocking tool is adapted to placed inside a lumen of a living body to block the lumen. The medical blocking tool includes a core section and a swelling section around the core section. The swelling section is comprised of a swelling material which swells in the presence of a liquid by absorbing the liquid so the volume of the swelling section increases. The swelling section is sized to be positioned inside the lumen of the living body. The swelling section swells when the medical blocking tool is positioned in the lumen in the presence of the liquid so that the swelling section engages the inside surface of the lumen of the living body to block the lumen.

With such a configuration, the medical blocking tool is transported to the portion (blocking target portion) to be blocked in the lumen of the body while the swelling section is in its contracted state (the state before the swelling operation), and is fixed (placed) thereto while the swelling section is swelled (expanded). Accordingly, it is possible to easily and promptly place the medical blocking tool.

In addition, since the medical blocking tool is fixed to the inside of the lumen of the body to block the lumen when the swelling section is swelled, it is possible to reliably fix the medical blocking tool, and to reliably block the lumen of the body.

For example, even when the surface of the body tissue defining the lumen of the body is uneven, since the medical blocking tool can reliably follow the shape of the unevenness, the medical blocking tool can be reliably adhered to the surface of the body tissue.

Because physical irritation of the swelling section with respect to the body tissue is relatively small, it is possible to suppress granulation or the like.

Also, because the swelling section of the medical blocking tool is in a contracted state before the medial blocking tool is placed inside of the lumen of the body, for example, when the lumen of the device (for example, a bronchoscope or the like when the medical blocking tool is placed inside of the bronchial tube) used to place the medical blocking tool is used, it is possible to transport the medical blocking tool to the front end of the device. Accordingly, it is possible to place the medical blocking tool at a plurality of positions while the medical blocking tool is inserted inside the body. Therefore, since it is possible to shorten the treatment time, it is possible to reduce the burden on the patient.

In the medical blocking tool with the above-described configuration, the swelling material is preferably a gel polymer.

Accordingly, the swelling material can be selectively swelled (expanded) or contracted in response to the characteristics of the contacting liquid.

In the medical blocking tool with the above-described configuration, a part of a molecular structure of the gel polymer preferably has an anionic group.

The swelling rate (expansion rate) and the contraction rate of the gel polymer are thus selectively increased in accordance with the concentration of contained ions, composition of contacting liquid or the like. In addition, the swelling rate (expansion rate) and the contraction rate of the environmentally-sensitive gel polymer are selectively increased in accordance with the concentration of contained ions, composition of contacting liquid or the like. Further, since the hydrophilic properties of the environmentally-sensitive gel polymer are increased, the gel polymer can be swelled by actively absorbing a larger amount of liquid. Furthermore, since the hydrophilic properties of the gel polymer are increased, the gel polymer can be swelled by actively absorbing a larger amount of liquid.

In the medical blocking tool with the above-described configuration, it is preferred that the volume of the gel polymer is increased when contacting a liquid deprotonating the anionic group, or the volume thereof is decreased when contacting a liquid protonating the anionic group.

The gel polymer is thus swelled when contacting a liquid exhibiting deprotonation, and is contracted when contacting a liquid exhibiting protonation.

The above-described medical blocking tool is preferably configured so that the gel polymer contains at least one type of monomer component selected from acrylic acid, methacrylic acid, and their derivatives.

Accordingly, since the gel polymer containing them has an anionic group, its environmental sensitivity is further improved.

In the medical blocking tool with the above-described configuration, the gel polymer preferably contains an ethylenically unsaturated compound as a cross-linking agent. It is thus possible to improve the mechanical characteristics of the gel polymer.

In the medical blocking tool with the above-described configuration, the core section may be formed as a bar-shaped wire rod, and the swelling section is preferably formed along the wire rod to coat the wire rod.

Accordingly, it is possible to quickly, easily, and reliably place the medical blocking tool inside of the lumen of the body.

The core section may be formed as a coil-shaped wire rod, and the swelling section is preferably formed as a coil shape along the wire rod to coat the wire rod.

When the swelling section is swelled, a gap in the swelling section is thus removed, so that the inside of the lumen of the body is blocked.

The core section may also be formed as a coil-shaped wire rod, and the swelling section is preferably formed as a bar shape to cover the entire portion of the wire rod.

The contact area between the core section and the swelling section is thus increased, and hence the adhesion between the core section and the swelling section is improved.

In the medical blocking tool with the above-described configuration, the core section preferably has a contrast property. Accordingly, it is possible to allow the core section (the medical blocking tool) to have X-ray contrast property, and to position the medical blocking tool inside of the bronchial tube while checking (watching) the position of the medical blocking tool using X-ray fluoroscopy.

The surface of the core section is preferably subjected to a roughing process. Accordingly, the contact area with respect to the swelling section is increased, and hence adhesion with respect to the swelling section is improved.

The medical blocking tool is preferably used to block a bronchial tube.

It is preferred that a fixation assisting means is further provided to assist fixation when the swelling section is fixed to the inside of the lumen of the body. This helps more reliably fix the medical blocking tool to the inside of the lumen of the body.

It is preferable that the fixation assisting means is formed of an elastic material, and the fixation assisting means is contracted when applying an external force against its elasticity, and is expanded when releasing the external force. Accordingly, it is possible to more reliably fix the medical blocking tool to the inside of the lumen of the body.

Therefore, it is possible to relatively quickly, easily, and reliably place the medical blocking tool inside of the bronchial tube.

According to another aspect, a medical blocking tool positionable inside a lumen of a living body to block the lumen of the living body includes a swelling section, a core section embedded in the swelling section, with the core section and the swelling section being made of different materials, and the swelling section having an outer surface which is an outer surface of the medical blocking tool. The swelling section possesses an outer dimension permitting the medical blocking tool to be positioned in the lumen of the living body, and the swelling section is comprised of a swelling material which swells to an enlarged size upon absorbing a first liquid and which contracts to a size smaller than the enlarged size when brought into contact with a second liquid different from the first liquid. The swelling section swells when the medical blocking tool is positioned in the lumen and is contacted by the first liquid so that the outer surface of the swelling section engages an inner surface of the lumen of the living body to block the lumen.

In accordance with another aspect, a method of blocking a lumen in a living body comprises: positioning a medical blocking tool inside the lumen of the living body tool, the medical blocking tool comprising a swelling section and a core section embedded in the swelling section, the swelling section possessing an outer surface and being made of a swelling material which swells when contacted by a liquid; moving the medical blocking tool in the lumen to position the medical blocking tool at a target blocking site in the lumen which is to be blocked, the lumen possessing an inner surface; and swelling the swelling section of the medical blocking tool by contacting the swelling section with the liquid to increase an outer dimension of the swelling section and produce a swelled swelling section in which the outer surface of the swelling section engages the inner surface of the lumen at the target blocking site to block the lumen.

### BRIEF DESCRIPTION OF THE DRAWINGS

FIG. 1 is a perspective view of a first embodiment of a medical blocking tool disclosed here.

FIG. 2 is a longitudinal cross-sectional view of the medical blocking tool shown in FIG. 1.

FIG. 3 is an explanatory illustration of the manner of using the medical blocking tool shown in FIG. 1.

FIG. 4 is another explanatory illustration of the manner of using the medical blocking tool shown in FIG. 1.

FIG. 5 is a further explanatory illustration of the manner of using the medical blocking tool shown in FIG. 1.

FIG. 6 is a side view of a second embodiment of the medical blocking tool disclosed here.

FIG. 7 is a side view of a third embodiment of the medical blocking tool disclosed here.

FIGS. 8A, 8B and 8C are longitudinal cross-sectional views illustrating a manner of using a fourth embodiment of the medical blocking tool disclosed here.

### DETAILED DESCRIPTION

The medical blocking tool disclosed here is useful as a medical blocking tools which is positionable inside a lumen of a body (to be fixed to body tissue defining the lumen of the body) and close the lumen of the body. The embodiments of the medical blocking tool disclosed here are described as being used, by way of example, as a bronchial tube blocking tool (bronchial tube closing tool) used to block (close) a bronchial tube.

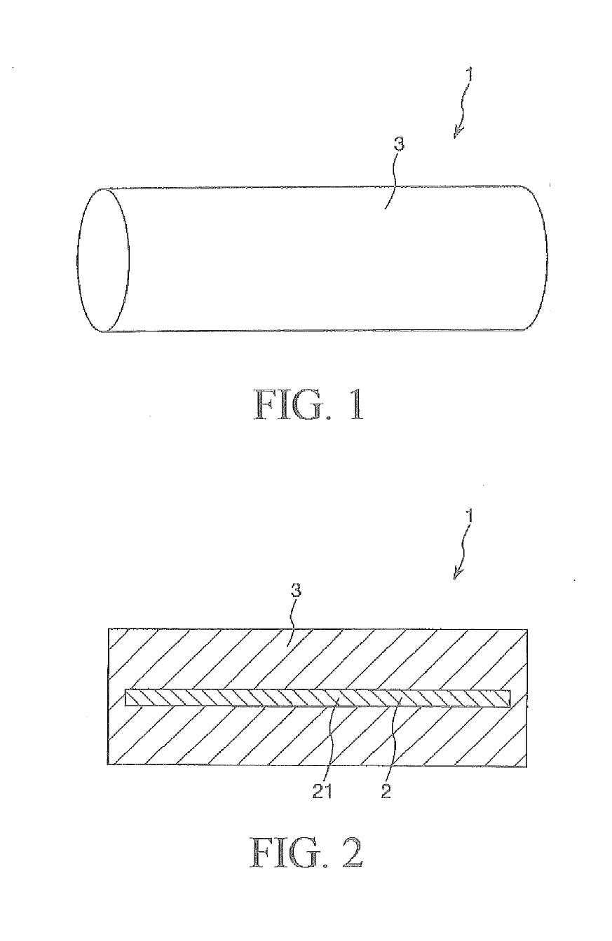

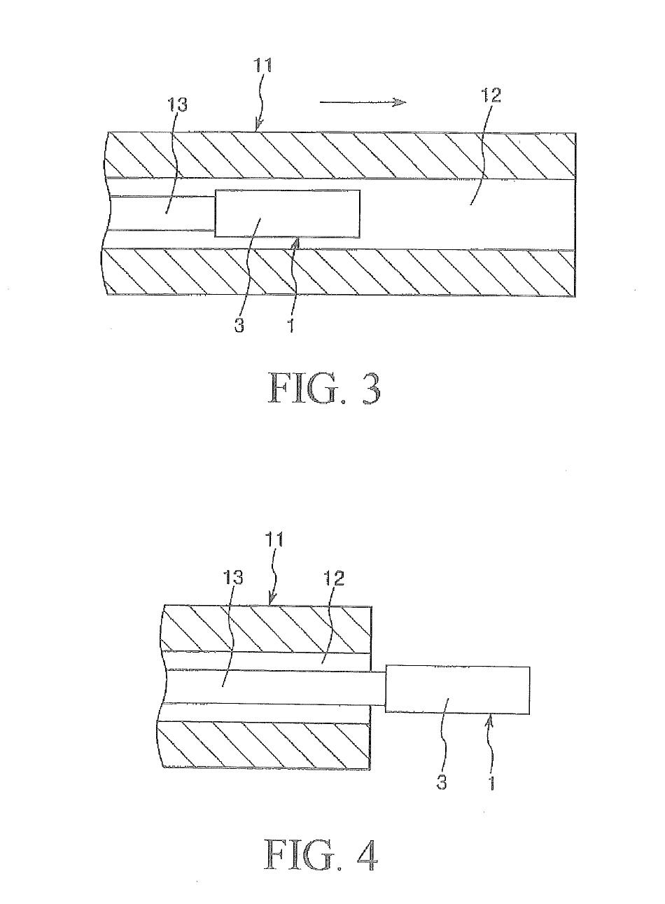

A first embodiment of the medical blocking tool disclosed here is shown in FIGS. 1 and 2, and an example of a manner of using (instructions for using) the medical blocking tool is illustrated in FIGS. 3-5.

In the description below, the right side in FIGS. 3 and 4 indicates the “front end” and the left side indicates the “base end ” or “rear end”.

The medical blocking tool 1 shown in the drawings is a tool which, by way of example, is positioned or placed inside the bronchial tube to block the bronchial tube.

The medical blocking tool 1 includes a core section 2 and a swelling section 3 provided (fixed) around the core section 2.

The core section 2 serves as a reinforcing member for reinforcing the swelling section 3 (the medical blocking tool 1). In the illustrated embodiment, the core section 2 is a bar-shaped wire rod 21, and possesses a linear shape. The core section 2 may be a solid member or a hollow member, and may be cylindrical in shape.

The outer surface of the core section 2 is preferably subjected to a roughening operation or process. This will help increase the contact area between the core section 2 and the swelling section 3, thus improving adhesion between the core section and the swelling section 3.

It is desirable that the core section 2 has a contrast property with respect to, for example, X-ray fluoroscopy (including CT scan), MRI, or the like. In addition, it is desirable that the core section 2 is formed of metal materials. Examples of metal materials forming the core section 2 include, for example, stainless steel, a hyperelastic alloy, a cobalt-based alloy, or a precious metal such as gold, platinum, and tungsten, or an alloy containing them (for example, platinum-iridium alloy). Particularly, when the core section 2 is formed of a radiopaque material such as a precious metal, that is, a material having a contrast property with respect to X-rays, it is desirable in that the contrast property with respect to the X-rays is obtained in the core section 2 (the medical blocking tool 1), and the medical blocking tool 1 can be placed inside the bronchial tube while checking (watching) the position of the medical blocking tool 1 under the X-ray fluoroscopy.

The dimensions (the length, the outer diameter, and the like) of the core section 2 are not particularly limited, and are appropriately determined in accordance with the portion (position) or case of placing the medical blocking tool 1. Generally, it is desirable that the length of the core section 2 is about 5 to 100 mm, and more desirable that the length is about 10 to 50 mm. In addition, it is desirable that the outer diameter of the core section 2 is about 0.01 to 1 mm, and more desirable that the outer diameter is about 0.02 to 0.5 mm.

The swelling section 3 is formed of a swelling material which swells (expands) by absorbing a liquid so that the volume of the material increases. The swelling section 3 is adapted to swell until the bronchial tube is blocked. This occurs by virtue of the swelling causing the outer surface of the tool to come into close contact with the inner wall or inner surface of the bronchial tube in the portion to be blocked (blocking target portion) in the bronchial tube. That is, the medical blocking tool 1 is fixed in position inside the bronchial tube as a result of the swelling operation of the swelling section 3 to thus block the bronchial tube. In the following description, it is assumed that the swelling section 3 is in a contracted state (a state before a swelling operation) unless otherwise stated.

The swelling section 3 is provided along the wire rod 21 of the core section 2, and coats the entirety of the wire rod 21 (core section 2). That is, the core section 2 is buried or embedded in the swelling section 3. In addition, in this embodiment, the swelling section 3 is bar-shaped in longitudinal cross-section, and the medical blocking tool 1 possesses a cylindrical shape.

The dimensions (the length, the outer diameter, etc.) of the swelling section 3 (medical blocking tool 1) are not particularly limited, and are appropriately determined based on the place at which the medical blocking tool 1 is to be positioned. Generally, it is desirable that the length of the swelling section 3 (medical blocking tool 1) is about 5 to 100 mm, more preferably about 10 to 50 mm.

In addition, the outer diameter of the swelling section 3 (medical blocking tool 1) is set to be smaller than the inner diameter of the lumen of a bronchoscope to be described later. This allows the medical blocking tool 1 to be movable in the inside of the lumen of the bronchoscope. The outer diameter of the swelling section 3 (medical blocking tool 1) is set so that the bronchial tube is blocked when the swelling section 3 is swelled. The outer diameter of the swelling section 3 (medical blocking tool 1) is preferably about 1 to 50 mm, more preferably about 5 to 30 mm.

The swelling material forming the swelling section 3 is not particularly limited so long as the swelling material is able to swell by absorbing a liquid so that the volume of the swelling section increases. A gel polymer is a desirable material that can be used for the swelling section 3. This embodiment will be described based on the assumption that a gel polymer is used as the swelling material for the swelling section.

The gel polymer (polymer) forming the swelling section 3 possesses or exhibits properties in which the volume of the section changes in response to contact with a liquid.

That is, the gel polymer in its dry state (most contracted state) before gelation is not in a gelation state, but swells (expands) to enter a gelation state when the inside of the gel polymer absorbs liquid. In addition, the gel polymer swelled in this way discharges the liquid which has been absorbed and enters a contracted state when the gel polymer comes into contact with a liquid having a predetermined property.

The gel polymer can thus be selectively swelled (expanded) or contracted in response to the properties of the contacting liquid. Such a gel polymer is generally referred to as “environmentally-sensitive gel polymer”.

The environmentally-sensitive gel polymer is mainly used as a cross-linker which is formed by polymerizing and linking environmentally-sensitive monomer components or pre-polymer components, and is composed of a polymer having a three-dimensional mesh structure.

Such an environmentally-sensitive gel polymer has void holes in gaps of molecular chains. The void holes are expanded and contracted in response to a binding force between the molecular chains, but the binding forces between the molecular chains change depending on the environment where the gel polymer exists. Due to such a property, the environmentally-sensitive gel polymer can absorb a liquid into the void holes, or discharge a liquid absorbed in the void holes in response to the change of the environment.

Examples of the monomer component or the pre-polymer component of the environmentally-sensitive gel polymer include acrylic acid, methacrylic acid, or derivatives thereof, styrenesulfonic acid, vinyl sulfonic acid, vinyl phosphoric acid, and one type of the above or mixture of two or more types of the above can be used.

Among these, acrylic acid, methacrylic acid, or such derivative is desirably used. Acrylic acid, methacrylic acid, or such derivative is the environmentally-sensitive monomer component (pre-polymer component), and the gel polymer containing them has an ionic functional group to be described later. Accordingly, the environmental sensitivity of the gel polymer is increased.

In addition, it is desirable that the monomer component or the pre-polymer component of the environmentally-sensitive gel polymer contains ethylenically unsaturated monomer such as 2-hydroxyethylacrylate, 2-hydroxyethylmethacrylate, acrylic amide, or methacrylic amide, or such derivative, in addition to the above-described examples.

Among them, particularly, acrylic amide is desirably used. By containing the acrylic amide, it is possible to improve the mechanical characteristics of the gel polymer.

In addition, a part of the molecular structure of the monomer component or the pre-polymer component has an ionic functional group. Accordingly, the swelling rate (expansion rate) and the contraction rate of the environmentally-sensitive gel polymer are selectively increased in accordance with the concentration of contained ions, composition of contacting liquid or the like. In addition, since the hydrophilic properties of the environmentally-sensitive gel polymer are increased, the gel polymer can be swelled by actively absorbing a larger amount of liquid.

The ionic functional group is an anionic group, and the environmentally-sensitive gel polymer containing such an ionic functional group is swelled by deprotonation, so that the volume thereof is increased. In addition, the swelled environmentally-sensitive gel polymer is contracted by protonation, so that the volume thereof is decreased. Accordingly, the environmentally-sensitive gel polymer containing the anionic group is swelled when contacting a liquid exhibiting deprotonation, and then is contracted when contacting a liquid exhibiting protonation.

That is, in its dry state (most contracted state) before gelation, the volume of the environmentally-sensitive gel polymer containing the anionic group is at its smallest. On the other hand, in the gelation state contacting a liquid, the swelling rate becomes larger and the volume increases as the pH of the contacting liquid becomes larger. Likewise, the volume of the environmentally-sensitive gel polymer containing the anionic group has a positive correlation with respect to the pH of the liquid contacting the gel polymer.

Examples of the anionic group include a carboxylic acid group, a mercapto group, a phosphoric acid group, a sulfonic acid group, and the like.

In addition, the ionic functional group may be a cationic group. In this case, the environmentally-sensitive gel polymer is swelled by protonation, so that the volume thereof is increased. In addition, the swelled environmentally-sensitive gel polymer is contracted by deprotonation, so that the volume thereof is decreased. Accordingly, the environmentally-sensitive gel polymer containing the cationic group is swelled when contacting a liquid exhibiting protonation, and then is contracted when contacting a liquid exhibiting deprotonation.

That is, in the dry state (most contracted state) before gelation, the volume of the environmentally-sensitive gel polymer containing the cationic group is at its smallest. On the other hand, in the gelation state contacting a liquid, the swelling rate becomes larger and the volume increases as the pH of the contacting liquid decreases. Likewise, the volume of the environmentally-sensitive gel polymer containing the cationic group has a negative correlation with respect to the pH of the liquid contacting the gel polymer.

Examples of the cationic group include an amino group, an ammonium group, and the like.

In the description below describing the manner of using the medical blocking tool 1 will discuss an example in which the ionic functional group is the anionic group.

The swelling material forming the swelling section 3 is not limited to the gel polymer. For example, a material may be used which swells by absorbing a liquid, but does not enter a gelation state.

A method of manufacturing the medical blocking tool 1 is now described.

[1] First, the raw material for forming the swelling section 3 is prepared.

A monomer component (pre-polymer component) as a raw material of the gel polymer, a cross-linking agent, a polymerization initiator, and a solvent are prepared. Then, a solution is prepared by mixing these materials. Accordingly, the monomer component is polymerized and linked stereoscopically, thereby obtaining a gel polymer having a three-dimensional mesh structure.

In addition, it is preferable that the content ratio of the environmentally-sensitive monomer component among the total monomer components is about 10 to 50 mass %, more preferably about 10 to 30 mass %.

The content ratio of the monomer component in the solution is not particularly limited, but is preferably about 20 to 30 mass %.

Examples of the cross-linking agent include ethylenically unsaturated compounds such as N,N′-methylenebisacrylamide, ethylene glycol di(meth)acrylate, diethylene glycol di(meth)acrylate, triethylene glycol di(meth)acrylate, polyethylene glycol di(meth)acrylate, trimethylolpropane tri(meth)acrylate, pentaerythritol tri(meth)acrylate, divinylbenzene, and divinylether. Since the ethylenically unsaturated compound functions as a cross-linking agent together with the monomer component to reliably form a three-dimensional mesh structure, the ethylenically unsaturated compound is particularly suitable as the cross-linking agent for forming the swelling section 3.

Among them, the N,N′-methylenebisacrylamide is more preferably used.

The content ratio of the cross-linking agent among the solution is not particularly limited. However, it is preferable that the content ratio is less than about 1 mass %, more preferably less than 0.1 mass %.

Examples of the polymerization initiator include ammonium persulfate, N,N,N′,N′-tetramethylethylene diamine, and the like.

Examples of the solvent include water, ethanol, and the like.

If necessary, a pore-forming agent may be contained in the solution. Accordingly, the swelling section 3 can have a porous structure. Since the swelling section 3 with such a porous structure has a relatively large surface area, the liquid absorption speed is relatively high, and the swelling speed (expansion speed) is relatively high.

Examples of the pore-forming agent include sodium chloride, potassium chloride, ice, saccharose, sodium bicarbonate, and the like.

In addition, it is preferable that the average particle diameter of the pore-forming agent is about 1 to 25 μm, more preferably about 3 to 10 μm.

The content ratio of the pore-forming agent in the solution is preferably about 5 to 50 mass %, more preferably about 10 to 20 mass %.

Furthermore, if necessary, particles formed of a material opaque to X-rays may be contained in the solution. Accordingly, the swelling section 3 can possess the X-ray contrast property, and the position of the swelling section 3 (the medical blocking tool 1) can be relatively easily checked or visually observed in the X-ray fluoroscopy.

[2] Subsequently, the obtained solution is coated on the core section 2, so that a gel polymer layer, that is the swelling section 3, is formed on the surface of the core section 2. The swelling section 3 of the medical blocking tool is thus produced, and the swelling section 3 in a swelled state.

[3] Subsequently, the swelling section 3 is cleaned by a cleaning liquid. Accordingly, unreacted residual monomer components, the pore-forming agent, and the like are removed.

[4] Subsequently, the swelling section 3 which is in the swelled state is dried. The swelling section 3 which is in a dry state (most or maximum contracted state) is formed, thereby producing the medical blocking tool 1.

Next, an example of the manner of using, or the instructions for use (operations), the medical blocking tool 1, that is the procedure of placing the medical blocking tool 1 inside the bronchial tube, is described.

As shown in FIG. 3, the medical blocking tool 1 is connected to the front end of an operation wire 13. The medical blocking tool 1 is preferably connected to the front end of the operation wire 13 in a separable manner. For example, the connection between the medical blocking tool 1 and the operation wire 13 can be a magnetic connection by installing an electromagnet at the front end of the operation wire 13. Alternatively, the medical blocking tool 1 and the operation wire 13 may be connected to each other at a connection portion which can be melted by heat. With this alternative, electrical current may be supplied to the connection portion between the medical blocking tool 1 and the operation wire 13 so that the connection portion is melted by generated heat. Alternatively, the operation wire 13 can be provided with a lumen, and this lumen of the operation wire 13 can be supplied with a liquid which is sprayed toward the medical blocking tool 1 connected to the operation wire 13 so that the medical blocking tool 1 is separated from the operation wire 13 by the pressure or force of the liquid.

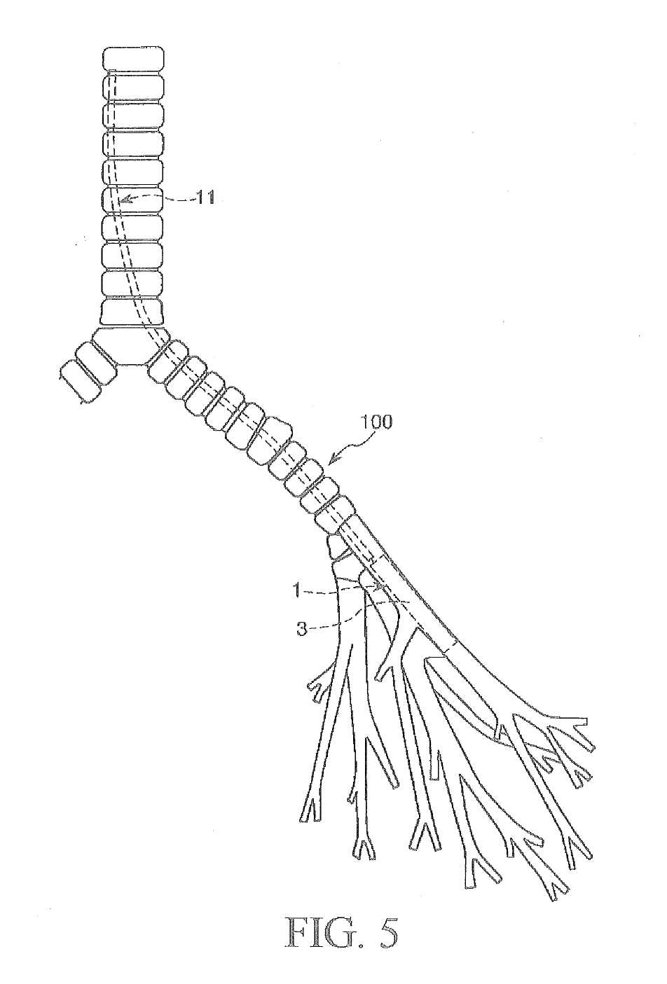

First, a bronchoscope 11 is inserted through a patient's mouth or nose, and the front end of the bronchoscope is positioned near the blocking target portion of a bronchial tube 100 as generally shown in FIG. 5.

Next, as shown in FIGS. 3 and 4, the medical blocking tool 1 is inserted into the lumen 12 of the bronchoscope 11, and the operation wire 13 is operated to be moved toward the front end of the lumen 12. Accordingly, the medical blocking tool 1 is moved in the inside of the lumen 12 of the bronchoscope 11 toward the front end of the bronchoscope, and is discharged from the front end of the lumen 12 to the blocking target portion of the bronchial tube 100.

As shown in FIG. 5, the swelling section 3 of the medical blocking tool 1 contacts the bodily fluids (tissue fluids) inside the bronchial tube 100, and the anionic group of the gel polymer forming the swelling section 3 is depronated, so that the swelling section 3 swells. Accordingly, the medical blocking tool 1 is fixed to the inside of the bronchial tube 100, and the blocking target portion of the bronchial tube 100 is blocked. In this case, for example, even if unevenness exists in the inner wall of the bronchial tube 100, the swelling section 3 can follow the uneven shape since the swelling section 3 is flexible or conformable. For this reason, the swelling section 3 can reliably contact and engage the inner wall of the bronchial tube 100. In addition, since the physical irritation of the swelling section 3 with respect to the inner wall of the bronchial tube 100 is relatively small, it is possible to suppress granulation or the like.

Here, a predetermined liquid may be supplied to the medical blocking tool 1 which has been positioned at the blocking target portion of the bronchial tube 100. The liquid can be supplied by way of the lumen of the bronchoscope 11. It is thus possible to relatively promptly and reliably swell the swelling section 3, and to swell the swelling section 3 at substantially the same time at all times. As the liquid supplied to the medical blocking tool 1, a liquid (for example, a buffer fluid) of which the pH is adjusted to be substantially equal to that of the bodily fluids inside the bronchial tube 100 is preferable.

Subsequently, the medical blocking tool 1 is separated from the front end of the operation wire 13. Accordingly, the medical blocking tool 1 is placed or positioned inside the bronchial tube 100.

Then, at the time when a medical blocking tool 1 is to be placed at another blocking target portion of the bronchial tube 100, the front end of the bronchoscope 11 is moved to the vicinity of the next blocking target portion of the bronchial tube 100, and the same operations as described above are performed. It is possible to place a plurality of medical blocking tools 1 at respective blocking target portions while the bronchoscope 11 remains inserted into the bronchial tube 100. It is thus possible to shorten the treatment time and reduce the burden on the patient.

In addition, the medical blocking tool 1 can be relatively easily extracted after placement inside the bronchial tube 100.

When it is desired to extract the medical blocking tool 1, a predetermined liquid is supplied to the medical blocking tool 1 placed inside the bronchial tube 100 by way of the lumen of the bronchoscope 11. The liquid that is used in this regard is a liquid (for example, a buffer fluid) whose pH is smaller than (less than) that of the bodily fluids inside the bronchial tube 100. Accordingly, the anionic group of the gel polymer forming the swelling section 3 is protonated, so that the swelling section 3 contracts. Accordingly, it is possible to relatively easily extract the medical blocking tool 1.

As described above, the medical blocking tool 1 disclosed here makes it possible to relatively easily and promptly place the medical blocking tool 1 at the blocking target portion of the bronchial tube, and to reliably block the blocking target portion.

The medical blocking tool 1 described above includes one core section, but it is to be understood that the medical blocking tool 1 can include a plurality of core sections.

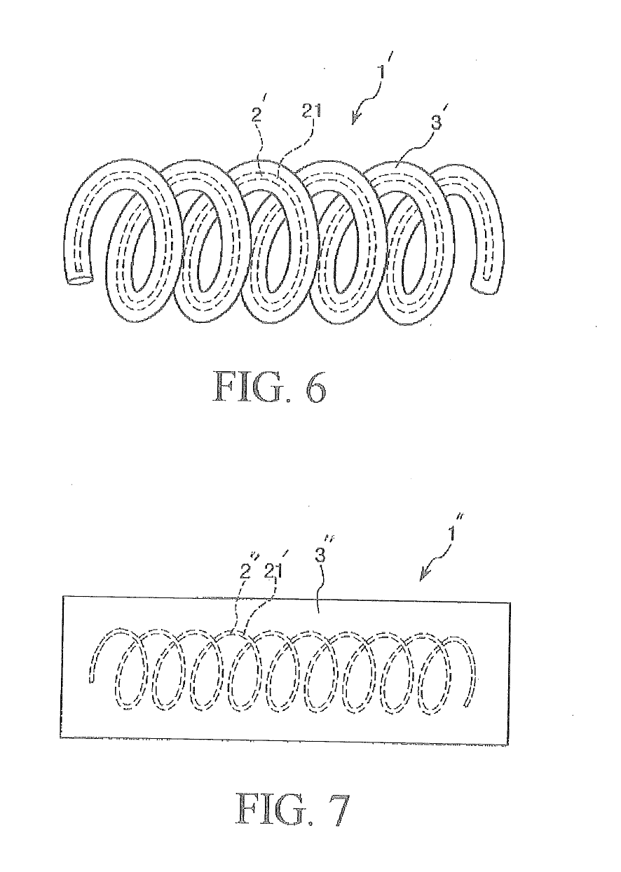

FIG. 6 illustrates a second embodiment of the medical blocking tool disclosed here. Features of this second embodiment of the medical blocking tool that are the same as features in the first embodiment are identified by the same reference numeral and a detailed description of those features is not repeated. The description below focuses primarily on aspects of the second embodiment that differ from the first embodiment.

As shown in FIG. 6, the medical blocking tool 1′ of the second embodiment includes a core section 2′ formed as a coil-shaped wire rod 21.

In addition, the swelling section 3′ is coil-shaped along the wire rod 21 of the core section 2′, and coats or covers the entire wire rod 21 (core section 2′). As shown in FIG. 4, the coil-shaped nature of the medical blocking tool 1′ (i.e., the wire rod 21 and the swelling section 3′) results in gaps between adjacent windings of the coil-shaped tool 1′ and generally along the axis of coil-shaped tool 1′. When the swelling section 3′ swells, the swelling eliminates those gaps because portions of the swelling section 3′ become enlarged and contact other enlarging portions of the swelling section 3′, The tool 1′ thus no longer possesses a coil-shape, but rather takes on more of a block-shape and so the bronchial tube is blocked by the tool 1′.

This embodiment of the medical blocking tool 1′ exhibits advantages similar to those discussed above in the first embodiment.

In this embodiment of the medical blocking tool 1′, the contact area between the core section 2′ and the swelling section 3′ is relatively large, and so the adhesion or connection between the core section 2′ and the swelling section 3′ is improved.

FIG. 7 illustrates a third embodiment of the medical blocking tool disclosed here. Features of this third embodiment of the medical blocking tool that are the same as features in embodiments described above are identified by the same reference numeral and a detailed description of those features is not repeated. The description below focuses primarily on aspects of the third embodiment that differ from the first embodiment.

As shown in FIG. 7, the medical blocking tool 1 of the third embodiment includes a core section 2″ in the form of a coil-shaped wire rod 21′.

In addition, the swelling section 3″ is bar-shaped and coats or covers the entire wire rod 21′ of the core section 2″.

This embodiment of the medical blocking tool 1 exhibits advantages similar to those discussed above in the first embodiment.

This embodiment of the medical blocking tool 1″ exhibits relatively large contact area between the core section 2″ and the swelling section 3″ is relatively large, and so the adhesion or connection between the core section 2″ and the swelling section 3″ is improved.

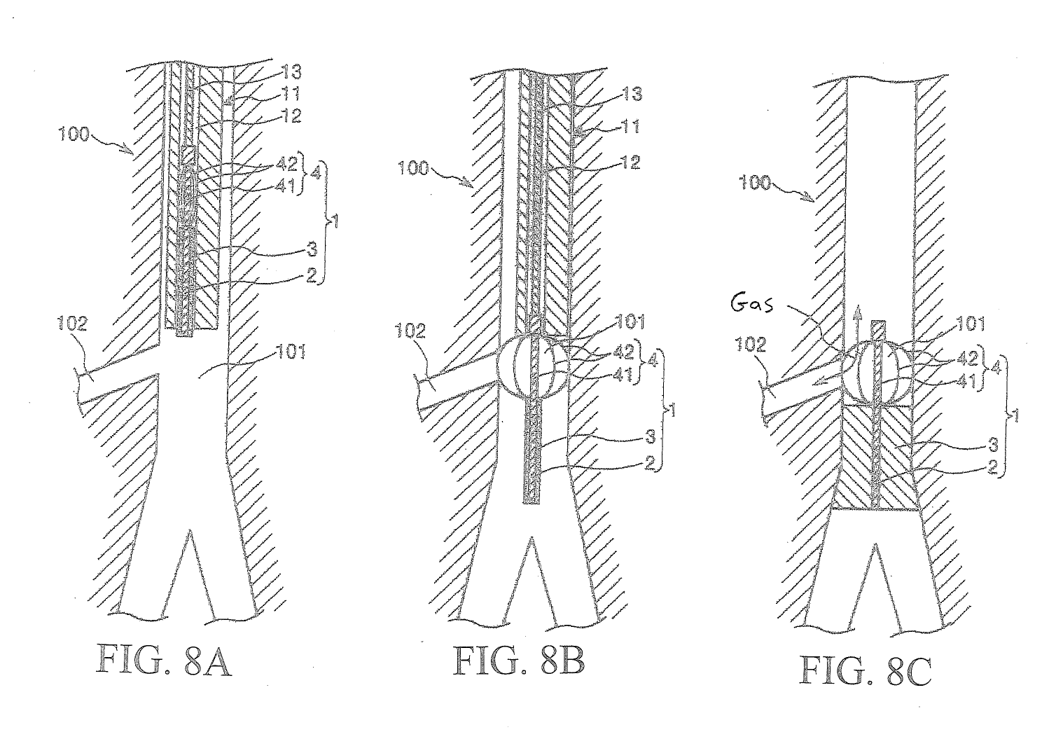

FIGS. 8A-8C illustrate a fourth embodiment of the medical blocking tool disclosed here. In the description which follows, the lower side in FIGS. 8A-8C is the “front end”, and the upper side is the “base end (rear end)”.

Features of this fourth embodiment of the medical blocking tool that are the same as features in embodiments described above are identified by the same reference numeral and a detailed description of those features is not repeated. The description below focuses primarily on aspects of the fourth embodiment that differ from the first embodiment.

This fourth embodiment of the medical blocking tool is the same as the first embodiment except that a fixation assisting means is further provided.

The medical blocking tool 1 shown in FIGS. 8A-8C includes a fixation assisting means 4. The fixation assisting means 4 is used to assist in fixing the swelling section 3 to the inside of the bronchial tube 100.

The fixation assisting means 4 includes an extension portion 41 which is disposed on the base end of the swelling section 3 and extends from the core section 2 to the base end, and a plurality of installation wires 42 installed or connected at both ends of the extension portion.

The extension portion 41 may be integrally formed with the core section 2. Alternatively, the extension portion 41 may be separately formed from the core section 2, and connected to the core section 2.

Each of the installation wires 42 is formed of an alloy which exhibits hyperelasticity inside the body. Accordingly, each of the installation wires 42 can be reliably deformed from the contracted state shown in FIG. 8A to the expansion state depicted in FIGS. 8B and 8C, and can have an accurate restored shape in the expansion state. The expansion state is the original state of the wires 42 and the wires 42 are restored to that shape inside the body. Here, an alloy which exhibits the hyperelasticity in the inside of the body means an alloy which has a property in which the alloy can be restored to substantially its original shape even when the alloy is deformed (bent, stretched, or compressed) up to an area where the composition of general metal is changed around at least a body temperature (around 37° C.). Examples of such alloys or materials include shape-memory alloys, hyperelastic alloys. The types of shape-memory alloys and hyperelastic alloys are not particularly limited, though titanium-based alloys (e.g., Ti—Ni, Ti—Pd and Ti—Nb—Sn) or copper-based alloys are preferable. An example of a desirable composition is one containing titanium of about 30 to 52 atom % and the remainder comprising nickel and one or more additional alloy components, wherein the one or more additional alloy components amount to 10 atom % or less.

In the medical blocking tool 1 (fixation assisting means 4) with such a configuration, when the medical blocking tool 1 is accommodated in the inside of the lumen 12 of the bronchoscope 11 as shown in FIG. 8A, each of the installation wires 42 is regulated (compressed) by the inner peripheral surface of the bronchoscope 11. That is, the inner peripheral surface of the bronchoscope 11 applies an inwardly directed external compressive force to the installation wire 42 in opposition to the elasticity of each of the installation wires 42. Accordingly, each of the installation wires 42 is contracted or compressed.

When the medical blocking tool 1 protrudes distally from the bronchoscope 11 as shown in FIGS. 8B and 8C, each of the installation wires 42 is expanded by its elasticity since the external force applied from the inner peripheral surface of the bronchoscope 11 is released. The fixation assisting means 4 is a so-called “self-expansion type” fixation assisting means.

As shown in FIGS. 8B and 8C, the expansion operation of the installation wire 42 is performed earlier than the swelling operation in which the swelling section 3 is expanded to be fixed to the bronchial tube 100. That is, the fixation performed by the expanded installation wire 42 and the fixation performed by the swelled swelling section 3 are carried out at different times spaced apart from one another by a time interval. Accordingly, the temporary fixation of the medical blocking tool is performed by the installation wire 42, and the actual fixation is performed by the swelling section 3. Therefore, it is possible to more reliably fix the medical blocking tool 1 in the bronchial tube 100.

The fixation assisting means 4 is configured so that the adjacent installation wires 42 are spaced from each other. Accordingly, as shown in FIGS. 8B and 8C, when the fixation assisting means 4 of the medical blocking tool 1 is positioned to a branch portion 101 of the bronchial tube 100, a gas can flow to branched portions 102 branched from the branch portion 101 via a gap between the adjacent installation wires 42. It is thus possible to ensure a bronchial pathway to the branched portions 102.

The fixation assisting means 4 is disposed on the base end side of the swelling section 3 in the illustrated embodiment, but other arrangements are possible. For example, the fixation assisting means 4 may be disposed on the front end side of the swelling section 3. Also, the expansion/contraction portion of the fixation assisting means 4 may be coil-shaped.

The fixation assisting means 4 is not limited to the self expansion type, but may be, for example, a balloon expansion type.

While the medical blocking tool of the invention has been described with reference to the embodiments shown in the drawings, the invention is not limited to such embodiments, and the configuration of the various features may be replaced by a differently configured feature which performs the same or a substantially similar function. In addition, other features can be added to the medical blocking tool.

The embodiments of the medical blocking tool may have a combination of features or characteristics from two or more embodiments.

The shape of the core section 2 is not limited to the above-described embodiments, but also possesses other shapes or configurations such as, for example, a particle shape (for example, a cubic shape, an elongated rectangular shape, or the like), a mesh shape (a lattice shape), or other shapes.

The medical blocking tool disclosed here is not limited to use in blocking the bronchial tube.

The medical blocking tool described here is placed inside the lumen of the body to block the lumen. The medical blocking tool includes the core section and the swelling section which is formed of a swelling material which swells by absorbing liquid so that the volume of the swelling material increases. The swelling section is provided around the core section, and when the swelling section swells, the swelling section is fixed to the inside of the lumen of the body to block the lumen. For this reason, it is possible to relatively quickly, easily, and reliably place the medical blocking tool inside the lumen of the body.

The detailed description above describes embodiments of the medical blocking tool for use in blocking a lumen. The invention is not limited, however, to the precise embodiment and variations described and illustrated above. Various changes, modifications and equivalents could be effected by one skilled in the art without departing from the spirit and scope of the invention as defined in the appended claims. It is expressly intended that all such changes, modifications and equivalents which fall within the scope of the claims are embraced by the claims.

What is claimed is:

1. A medical blocking tool positionable inside a lumen of a living body to block the lumen of the living body, the medical blocking tool comprising:
a swelling section;
a core section embedded in the swelling section, the core section and the swelling section being made of different materials;
the swelling section having an outer surface which is an outer surface of the medical blocking tool;
the swelling section possessing an outer dimension permitting the medical blocking tool to be positioned in the lumen of the living body;
the swelling section being comprised of a swelling material which swells to an enlarged size upon absorbing a first liquid and which contracts to a size smaller than the enlarged size when brought into contact with a second liquid different from the first liquid; and
the swelling section swelling when the medical blocking tool is positioned in the lumen and is contacted by the first liquid so that the outer surface of the swelling section engages an inner surface of the lumen of the living body to block the lumen.

2. The medical blocking tool according to claim 1, wherein the swelling material is a gel polymer possessing a molecular structure, at least a part of the molecular structure of the gel polymer comprising an anionic group.

3. The medical blocking tool according to claim 1, wherein the core section is a bar-shaped wire rod, and the swelling section is formed along the wire rod and coats the wire rod.

4. The medical blocking tool according to claim 1, wherein the core section is a coil-shaped wire rod, and the swelling section extends along the wire rod and is also coil-shaped.

5. The medical blocking tool according to claim 1, wherein the core section is a coil-shaped wire rod, and the swelling section is bar-shaped and entirely covers the coil-shaped wire rod.

6. The medical blocking tool according to claim 1, further comprising fixation assisting means for assisting fixation of the medical block tool in the lumen before the swelling section swells and engages the inner surface of the lumen of the living body.

7. A medical blocking tool positionable inside a lumen of a living body to block the lumen of the living body, the medical blocking tool comprising:
a core section;
a swelling section around the core section;
the swelling section being comprised of a swelling material which swells in a presence of a liquid by absorbing the liquid so a volume of the swelling section increases;
the swelling section being sized to be positioned inside the lumen of the living body; and
the swelling section swelling when the medical blocking tool is positioned in the lumen in the presence of the liquid so that the swelling section engages an inside surface of the lumen of the living body to block the lumen.

8. The medical blocking tool according to claim 7, wherein the swelling material is a gel polymer.

9. The medical blocking tool according to claim 8, wherein the gel polymer possesses a molecular structure, at least a part of the molecular structure of the gel polymer comprising an anionic group.

10. The medical blocking tool according to claim 9, wherein a volume of the gel polymer increases when contacting a liquid deprotonating the anionic group, and the volume of the gel polymer decreases when contacting a liquid protonating the anionic group.

11. The medical blocking tool according to claim 7, wherein the core section is a bar-shaped wire rod, and the swelling section is formed along the wire rod and coats the wire rod.

12. The medical blocking tool according to claim 7, wherein the core section is a coil-shaped wire rod, and the swelling section extends along the wire rod and is also coil-shaped.

13. The medical blocking tool according to claim 7, wherein the core section is a coil-shaped wire rod, and the swelling section is bar-shaped and entirely covers the coil-shaped wire rod.

14. The medical blocking tool according to claim 7, wherein the core section is made of a material possessing contrast properties.

15. The medical blocking tool according to claim 7, wherein an outer surface of the core section is roughened.

16. The medical blocking tool according to claim 7, further comprising fixation assisting means for assisting fixation of the medical block tool in the lumen before the swelling section swells and engages the inner surface of the lumen of the living body.

17. The medical blocking tool according to claim 16, wherein the fixation assisting means comprises an elastic material possessing elasticity, and wherein the fixation assisting means contracts when an external force is applied in opposition to the elasticity of the elastic material, and is expanded upon releasing the external force.

18. A method of blocking a lumen in a living body comprising:
positioning a medical blocking tool inside the lumen of the living body tool, the medical blocking tool comprising a swelling section and a core section embedded in the swelling section, the swelling section possessing an outer surface and being made of a swelling material which swells when contacted by a liquid;
moving the medical blocking tool in the lumen to position the medical blocking tool at a target blocking site in the lumen which is to be blocked, the lumen possessing an inner surface; and
swelling the swelling section of the medical blocking tool by contacting the swelling section with the liquid to increase an outer dimension of the swelling section and produce a swelled swelling section in which the outer surface of the swelling section engages the inner surface of the lumen at the target blocking site to block the lumen.

19. The method according to claim 18, wherein the liquid is a first liquid delivered to the swelling section after the medical blocking tool is positioned at the target blocking site.

20. The method according to claim 18, further comprising delivering a second liquid to the swelling section to contract the swelled swelling section, the second liquid being different from the first liquid.
